SEQUENCE LISTING (4 sequences)

SEQ ID NO: 1 - DNA, 38 bp, Homo sapien
gagactcccatggatccactgcctgtggggaatcctgc

SEQ ID NO: 2 - DNA, 24 bp, Homo sapien
ttagaagcggaattgtgcatttac

SEQ ID NO: 3 - DNA, 20 bp, Homo sapien
ctgcctgtggggaatcctgc

SEQ ID NO: 4 - DNA, 6 bp, Homo sapien
ggatcc

(19) United States
(12) Patent Application Publication
(10) Pub. No.: US 2005/0208123 A1
(43) Pub. Date: Sep. 22, 2005

(54) Chylamydia vaccine

(75) Inventors: Jean-Francois Lucien Maisonneuve, Seattle, WA (US); Jean-Paul Prieels, Rixensart (BE); Moncef-Mohamed Slaoui, Rixensart (BE); Vincent Georges Christian Louis Verlant, Rixensart (BE)

Correspondence Address: GLAXOSMITHKLINE;Corporate Intellectual Property - UW2220, P.O. Box 1539, King of Prussia, PA, 19406-0939, US

(73) Assignee: SmithKline Beecham Biologicals, sa

(21) Appl. No.: 11/088,301

(22) Filed: Mar. 24, 2005

Related U.S. Application Data
(63) Continuation of application No. 10/289,361, filed on Nov. 6, 2002, now abandoned.
Application No. 10/289,361 is a continuation of application No. 09/657,473, filed on Sep. 7, 2000, now abandoned.
Application No. 09/657,473 is a continuation-in-part of application No. 09/331,533, filed on Jun. 23, 1999, now abandoned.
Application No. 09/657,473 is a continuation-in-part of application No. 08/930,729, filed on Mar. 19, 1998, now abandoned.

(30) Foreign Application Priority Data
Apr. 3, 1995 (GB) 9506863.1
Dec. 24, 1996 (GB) 9626864.4

(51) Int. Cl. A61K 39/02; A61K 39/118; A61K 9/127

(52) U.S. Cl. 424/450; 424/190.1; 424/263.1

(57) ABSTRACT

Vaccine preparations are provided for the prevention of Chylamydia infections comprising a major outer membrane protein from chlamydia and a mucosal adjuvant such as a combination of QS21 and 3D-MPL, or chlorea Toxin or Heat labile enterotoxin. Such preparations provide protection from Chlamydia induced infertility.

### CROSS REFERENCE TO RELATED APPLICATIONS

This is a continuation-in-part of application Ser. No. 08/930,729 filed Mar. 19, 1998 and application Ser. No. 09/331,533 filed Jun. 23, 1999, the contents of which are incorporated herein by reference.

### BACKGROUND OF THE INVENTION

The obligate intracellular gram-negative bacterium Chlamydia trachomatis is a common human pathogen which infects mucosal epithelial cells of the conjunctiva and of the urogenital tract, causing a wide spectrum of human diseases such as trachoma and genital infections which can result in long term sequelae. Trachoma, which is endemic in several developing countries, is the world's leading cause of preventable blindness. Genital chlamydial infections are the most common bacterial sexually transmitted diseases (STD) in the US, representing around 3 million cases per year and rendering annually 200,000 women infertile following Chlamydia salpingitis (Washington, et al., JAMA, 257:2070-2072, 1987). The infection exerts its most detrimental consequences in women, the cervix being the most commonly infected site although severe complications like endometritis, pelvic inflammatory diseases (PID) and salpingitis can result from ascending infections leading to infertility and ectopic pregnancy. It has been shown that, whereas a single episode of PID can result in an infertility rate of 6.1%, three or more episodes have led to an infertility rate of 54% (Pickett, et al., Molecular Microbiology, 2:681-685, 1988).

Therefore, this pathogen is a significant public health problem and efforts are made to set up a vaccine against human Chlamydia infections.

Vaccine trials performed in man and non-human primates using the whole organism as immunogen gave serovar-specific protection but some of the vaccinees developed more severe reactions upon reinfection (Grayston, et al., The Journal of Infectious Diseases, 132:87-105, 1975). Several studies have demonstrated that the pathology associated with Chlamydia infection is immunologically mediated (Grayston, et al., Reviews of Infectious Diseases, 7:717-725, 1985); moreover, a purified Chlamydia 57 kDa (Hsp60) was shown to elicit a pathology similar to reinfection in animals previously infected (Morrison, et al., J. Exp. Med., 170:1271-1283, 1989; Blander, et al., Infect. Immun., 62:3617-3624, 1994). These observations led to the conclusion that protection against Chlamydia trachomatis could only be achieved using a subunit vaccine.

The Chlamydia trachomatis species is stereotyped into 15 serovars which are placed into 3 serogroups: the B complex (serovars B, Ba, D, E, L1 and L2), the intermediate complex (serovars F, G, K, L3) and the C complex (serovars A, C, H, I and J) (Wang, et al., The Journal of Infectious Diseases, 152:791-800, 1985). Sexually transmitted diseases are caused by serovars D to K which cover the 3 serogroups. Thus a subunit vaccine against Chlamydia STD should protect against multiple serovars that are more or less antigenically related.

For the design of a subunit vaccine, much interest has been focused on the serotyping antigen which consist in the 40 kDa major outer membrane protein (MOMP). This protein which was shown to function in vitro as a porin (Bavoil, et al., Infect. Immun., 44:479-485, 1984), is present during the whole life cycle of the bacteria (Hatch, et al., J. Bacteriol., 165:379-385, 1986) and this principal surface protein is highly immunogenic in humans and animals. The MOMP display 4 variable domains (VD) surrounded by five constant regions that are highly conserved among serovars (Stephens, et al., J. Bacteriol., 169:3879-3885, 1987; Yuan, et al., Infect. Immun., 57:1040-1049, 1989). In vitro and in vivo neutralizing B-cell epitopes have been mapped on VDs (Baehr, et al., Proc. Natl. Acad. Sci. U.S.A., 85:4000-4004, 1988; Lucero, et al., Infect. Immun., 50:595-597, 1985; Zhang, et al., J. Immunol., 138:575-581, 1987; Peterson, et al., Infect. Immun., 56:885-891, 1988; Zhang, et al., Infect. Immun., 57:636-638, 1989) whereas T-cell epitopes have been identified in both variable and constant domains (Allen, et al., J. Immunol., 147:674-679, 1991; Su, et al., J. Exp. Med., 172:203-212, 1990). The protein is produced with a signal sequence which is cleaved to produce the full-length mature protein. Recombinant MOMP has been expressed in E. coli by different authors (Manning, et al., Infect. Immun., 61:4093-4098, 1993; Koehler, et al., Molecular Microbiology, 6:1087-1094, 1992; Pickett, et al., Molecular Microbiology, 2:681-685, 1988); however, Manning et al. have shown that their recombinant protein failed to react with a monoclonal antibody that recognize a conformational MOMP epitope (Manning, et al., Infect. Immun., 61:4093-4098, 1993).

Immunizations with recombinant or purified MOMP followed by homotypic or heterotypic Chlamydia challenge have been performed in different animal models with variable effects on the parameters of the infection (Taylor, et al., Investigative Ophthalmology and Visual Science, 29:1847-1853, 1988; Batteiger, et al., Journal of General Microbiology, 139:2965-2972, 1993; Tuffrey, et al., Journal of General Microbiology, 138:1707-1715, 1992). An elegant experimental model of salpingitis has been developed in mice in which intrauterine inoculation of a human strain of Chlamydia trachomatis leads to long term infertility (Tuffrey, et al., Br. J. Exp. Path., 67:605-616, 1986; Tuffrey, et al., Br. J. Exp. Path., 78:251-260, 1986). In a heterotypic challenge experiment, Tuffrey et al. have shown that parenteral and mucosal immunization with rMOMP absorbed on alhydrogel reduced the severity of the salpingitis and the duration of the lower genital tract colonization, respectively. However, the preparation conferred no protection against infertility resulting from infection (Tuffrey, et al., Journal of General Microbiology, 138:1707-1715, 1992).

Both cell mediated and humoral immunity seem to play a protective role in the genital pathologies caused by Chlamydia trachomatis. However, Rank's group suggests that in mice T-cell mediated immunity is the principal immune mechanism for controlling chlamydial genital disease (Ramsey, et al., Infect. Immun., 56:1320-1325, 1988; Rank, et al., Infect. Immun., 48:847-849, 1985; Igietseme, et al., Infect. Immun., 59:1346-1351, 1991) and CD4 and CD8 positive T-cells have been shown to contribute to anti-chlamydial immunity in vivo (Igietseme, et al., Regional Immunology, 5:317-324, 1993; Igietseme, et al., Infect. Immun., 62:5195-5197, 1994). It has been shown that adoptive transfer of a MoPn-specific Th1 clone enables infection to be resolved in nude mice, genitally infected with MoPn. The activation of a predominantly Th1-like subset is consistent also with the protective immune response to other intracellular pathogens such as Leishmania (Heinzel, et al., J. Exp. Med., 169:59-72,1989) and Mycobacterium (Yamamura, et al., Science, 254:277-279, 1991).

### BRIEF SUMMARY OF THE INVENTION

The present invention relates to a vaccine formulation capable of providing protection against Chlamydia infections and in particular against the sequelae of the disease. In particular, to a formulation containing recombinant or purified major outer membrane protein from Chlamydia trachomatis combined with a mucosal adjuvant, that induces a MOMP-specific Th1 T cell immune response. More particularly the invention relates to a formulation containing a MOMP from Chlamydia adjuvanted with QS21 and 3D-MPL or a mutated heat-labile enterotoxin (mLT) from E. coli or cholera toxin (CT). Furthermore, the present invention relates to methods of preventing and/or treating chlamydia infections comprising administering to a patient in need the vaccine formulation of the present invention.

### DETAILED DESCRIPTION OF THE INVENTION

The present invention provides a vaccine composition which is effective at the mucosal level in conferring protection against infertility resulting from Chlamydia infections. Advantageously, the vaccine is effective in the mucosa where Chlamydia infections are primarily associated. The vaccine may be administered by any known route, including systemic or mucosal, but is advantageously useful as a mucosal vaccine, preferably as an oral or intranasal vaccine.

Accordingly the present invention provides a vaccine formulation comprising a recombinant or purified major outer protein (rMOMP) and a mucosal adjuvant. In particular, the vaccine contains MOMP from the B complex serogroup, more specifically MOMP from the serovar L2, D or E, but may also contain antigens from other serovars such as serovar F. Preferably the vaccine contains at least a MOMP from serovar L2. Combination vaccines comprising MOMP from two or more serovars may be utilised. A preferred combination comprises at least a MOMP from L2 serovar, additionally containing antigens from other serovars, such as D and E serovars. Another preferred combination comprises a MOMP from D serovar additionally comprising antigens from serovars E or L2. Yet another preferred combination comprises a MOMP from E serovar additionally comprising antigens from serovars D or L2.

In preferred compositions of the invention, the mucosal adjuvant is a combination of QS21 and 3 De-O-acylated monophosphoryl lipid A (3D-MPL). Other preferred compositions of the present invention contain as a mucosal adjuvant a mutated LT (for example LT R192G) from E. coli or the cholera toxin (CT). Mutated LT R192G can be obtained from following the teaching of IPA PCT/US95/09005 published under Ser. No. 96/06627. Cholera Toxin is available commercially from Swiss Serum, Bern.

Accordingly the present invention provides a vaccine formulation comprising 3D-MPL, QS21 and MOMP from Chlamydia. Alternatively, the present invention provides a vaccine formulation comprising a MOMP from Chlamydia combined with mLT or CT.

3 De-O-acylated monophosphoryl lipid A is known from GB2 220 211 (Ribi). Chemically it is a mixture of 3-deacylated monophosphoryl lipid A with 4, 5 or 6 acylated chains and is manufactured by Ribi Immunochem Montana.

QS21 is a Hplc purified non toxic fraction of a saponin from the bark of the South American tree Quillaja saponaria molina and its method of its production is disclosed (as QA21) in U.S. Pat. No. 5,057,540. Vaccines comprising both QS21 and 3D-MPL are disclosed in International Patent Application No. WO 94/00153.

In a preferred embodiment, QS21 is presented with a sterol since such compositions show decreased reactogenicity and improves the stability of QS21 to base-mediated hydrolysis.

In preferred compositions of the invention, the QS21 is associated with liposome structure containing cholesterol (hereinafter referred to as DQ). Such adjuvant compositions are described in copending UK Patent Applications 9513107.4 and WO 96/33739 whose disclosure is incorporated herein by reference. The antigen and the 3D-MPL are, in this preferred formulation, outside the structure of the liposome.

Vaccine preparation is generally described in New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in each vaccine dose is selected as an amount which induces an immunoprotective response without significant, adverse side effects in typical vaccines. Such amount will vary depending upon which specific immunogen is employed and how it is presented. Generally, it is expected that each dose will comprise 1-1000 μg of protein, preferably 2-100 μg, most preferably 4-40 μg. An optimal amount for a particular vaccine can be ascertained by standard studies involving observation of appropriate immune responses in subjects. Following an initial vaccination, subjects may receive one or several booster immunisation adequately spaced.

The vaccine formulation may be applied to a mucosal surface of a mammal in either a priming or boosting vaccination regime; or alternatively, it may be administered systemically. The mucosal route may include intranasal, oral, rectal or vaginal route. The systemic or parenteral route may include intramuscular, intradermal, transdermal, subcutaneous, intraperitoneal or intravenous administration. A preferred route of administration is via the transdermal route, for example by skin patches.

The formulations of the present invention maybe used for both prophylactic and therapeutic purposes.

Accordingly in one aspect, the invention provides a method of treatment comprising administering, through the mucosal or the parenteral route, an effective amount of a vaccine of the present invention to a patient.

Accordingly, the vaccine preparations of the present invention may be used to protect or treat a mammal susceptible to, or suffering from Chlamydia infection, by means of administering said vaccine by intramuscular, intraperitoneal, intradermal, transdermal, intravenous, or subcutaneous administration. Methods of systemic administration of the vaccine preparations may include conventional syringes and needles, or devices designed for ballistic delivery of solid vaccines (WO 99/27961), or needleless pressure liquid jet device (U.S. Pat. No. 4,596,556; U.S. Pat. No. 5,993,412), or transdermal patches (WO 97/48440; WO 98/28037). The present invention may also be used to enhance the immunogenicity of antigens applied to the skin (transdermal or transcutaneous delivery WO 98/20734 ; WO 98/28037). The present invention, therefore, further provides a delivery device for systemic administration, pre-filled with the vaccine composition of the present invention.

Apart from bypassing the requirement for painful injections and the associated negative effect on patient compliance because of “needle fear”, mucosal vaccination is attractive since it has been shown in animals that mucosal administration of antigens has a greater efficiency of inducing protective responses at mucosal surfaces, which is the route of entry of many pathogens. In addition, it has been suggested that mucosal vaccination, such as intranasal vaccination, may induce mucosal immunity not only in the nasal mucosa, but also in distant mucosal sites such as the genital mucosa (Mestecky, Journal of Clinical Immunology, 7:265-276, 1987). More advantageously, besides its superiority in inducing mucosal immune responses, one attractive advantage of the mucosal vaccination relies on its ability to also induce good systemic immunity. The non-parenteral administration of vaccines may therefore be an efficient and more convenient way to boost systemic immunity induced by parenteral vaccination, especially when multiple boosts are required to sustain a vigorous systemic immunity.

Alternatively the vaccine preparations of the present invention may be used to protect or treat a mammal susceptible to, or suffering from disease, by means of administering said vaccine via a mucosal route, such as the oral/alimentary or nasal route. Alternative mucosal routes are intravaginal and intra-rectal. The preferred mucosal route of administration is via the nasal route, termed intranasal vaccination. Methods of intranasal vaccination are well known in the art, including the administration of a droplet, spray, or dry powdered form of the vaccine into the nasopharynx of the individual to be immunised. Nebulised or aerosolised vaccine formulations also form part of this invention. Enteric formulations such as gastro resistant capsules and granules for oral administration, suppositories for rectal or vaginal administration also form part of this invention. The vaccines of the present invention may also be administered via the oral route. In such cases the pharmaceutically acceptable excipient may also include alkaline buffers, or enteric capsules or microgranules. The vaccines of the present invention may also be administered by the vaginal route. In such cases, the pharmaceutically acceptable excipients may also include emulsifiers, polymers such as CARBOPOL®, and other known stabilisers of vaginal creams and suppositories. The vaccines of the present invention may also be administered by the rectal route. In such cases the excipients may also include waxes and polymers known in the art for forming rectal suppositories.

In an embodiment of the invention the MOMP antigen is from serovar L2 or serovar D or serovar E and is produced in E. Coli by means of recombinant DNA techniques. In such circumstances the protein is produced without its signal sequence as a full-length mature protein.

In the present invention, adjuvantation of the antigen with or without 3D-MPL+QS21 strongly influenced the IgG1:IgG2a ratio in immunized groups; immunization with 3D-MPL+QS21 was associated with low IgG1:IgG2a ratios and partial protection while immunization without 3D-MPL+QS21 led to higher IgG1:IgG2a ratio and gave no protection. Interestingly, the switching to IgG2a antibody production by B cell is mediated by gamma interferon which are produced by the Th1 subset of T-helper lymphocytes whereas IgG1 production is mediated by interleukin-4 secreted by Th2 cells (Snapper, et al., Science, 236:944-947, 1987). As IgG2a production is controlled by Th1 cell products, it would seem likely that the protected groups presented a 3D-MPL+QS21 driven Th1 cell activation. In human and in mouse models, Th1 cytokines, IL-2 and IFN-gamma, are generally associated with resistance to infection with intracellular pathogens whereas Th2 cytokines, IL-4 and IL-10, are associated with progressive disease. This can be illustrated with the resistance or susceptibility of inbred strains of mice to Leishmania major that correlates with the induction of specific Th1 or Th2 response (Heinzel, et al., Proc. Natl. Acad. Sci. U.S.A., 88:7011-7015, 1991). Moreover, interferon gamma has anti-chlamydial activity in vitro and is involved in resolving the infection in vivo (Byrne, et al., Infect. Immun., 53:347-351, 1986; Rank, et al., Infect. Immun., 60:4427-4429, 1992). Thus, immunostimulants driven Th1 cytokines could be responsible for the protection observed in this vaccination experiment.

Two other observations support the hypothesis of the installation of a protective cell-mediated immunity in the groups vaccinated through the systemic route such as rMOMP+3D-MPL+QS21 and rMOMP+3D-MPL DQ groups: firstly, the absence of specific secretory IgA in the vaginal secretions and the non neutralizing nature of the strong seric antibody response rule out the possibility of having a humoral protection; secondly, the heterotypic character of the protection obtained using two distinct serovars differing considerably at the level of the MOMP VDs sequence suggests that T-cell epitopes from processed conserved domains of MOMP could be the agent of the protection against infertility.

Similarly to the results obtained with QS21 and 3D-MPL combination, we have also generated evidence that mucosal immunisation with rMOMP combined with CT or mLT can afford protection against infertility caused by Chlamydial challenge.

The following examples illustrate the invention.

A. Set of Experiments Performed with QS21+3D-MPL Combination as Adjuvant

A 1. Material and Methods

A 1.1 Chlamydial Strains and Animals

Chlamydia trachomatis serovar F strain NI1 isolated by Tuffrey et al. and kindly provided by Dr. J. Orfila and Chlamydia trachomatis serovar L2 strain 434 (ATCC, Rockville, Md.) were used in this study. Chlamydia were inoculated on Mc Coy cells (ATCC, Rockville, Md.) at a concentration of approximately 10⁶ inclusion forming units/ml (IFU/ml) in MEM supplemented with 10% foetal calf serum (FCS) (Gibco BRL). After 1 h centrifugation (1500 g) and 2 h incubation at 37° C. (5% CO2), the inoculum was removed and cell were refed with fresh medium supplemented with 0.5 μg/ml cycloheximide (Sigma). After incubation at 37° C. for 48 h, cells were disrupted with glass beads, harvested in 250 mM sucrose, 10 mM sodium phosphate, 5 mM L-glutamic acid pH 7.2 (SPG) to an approximate concentration of 10⁷ to 10⁸ IFU/ml and stored at −70° C.

Female C3H/HeOuJ (H-2^{k}) mice, 6-8 weeks old were obtained from Iffa Credo (France). 8 to 10 weeks old males from the same strain (B & K , U.K.) were used for breeding.

A 1.2 PCR Amplification and Plasmid Constructions

Amplification.

MOMP serovar L2 DNA was obtained by lysis of 10 μl of the chlamydial inoculum in 240 μl of lysis buffer and PCR amplification as described previously by Denamur et al. (40). Synthetic oligonucleotides

[SEQ ID NO:1]

5′-GAGACTCCCATGGATCCACTGCCTGTGGGGAATCCTGC-3′

and

| | |
|---|---|
| 5′-TTAGAAGCGGAATTGTGCATTTAC-3′ | [SEQ ID NO:2] |

(SB Biologicals, Belgium) were chosen from the published sequence (Zhang, et al., Nucleic Acids Research, 18:1061, 1990). The 5′ oligonucleotide contained the nucleotide sequence coding for the amino-terminus of the mature MOMP (underlined) preceded by a BamHI restriction endonuclease site (bold type). The amplification was carried out using Pfu DNA polymerase (Stratagen) and a Koch Light NBS Thermal Cycler (New Brunswick). A right sized PCR product was purified from a 1% agarose gel using a Geneclean II kit (Bio 101).

A 1.3 Cloning

The amplified serovar L2 MOMP DNA was rendered blunt-end with the Klenow fragment of DNA polymerase I, ligated into pGEM4Z (Promega) previously digested with SmaI and transformed into E. coli JM109 using the standard CaCL₂ protocol. Restriction analysis was performed on the resulting clones and a right DNA construct was amplified and purified using a nucleobond PC-100 kit (Macherey-Nagel). MOMP DNA was then excised from pGEM4Z-MOMP by digestion with BamHI and inserted into BamHI digested pET15 (Novagen) downstream the T7lac promotor and the His.Tag sequence. Right clones were selected by restriction analysis after transformation into the E. coli strain DH10B (stratagen); the complete nucleotide sequence of the cloned DNA was verified by the dideoxy chain termination method (Tabor, et al., Proc. Natl. Acad. Sci. U.S.A., 86:4076-4080, 1989). A pET15-MOMP plasmid preparation was finally used to transform the BL21(DE3) strain (Novagen) which is able to promote the recombinant product expression as it possess an IPTG inducible T7 polymerase.

A 1.4 Immunogen Production, Characterization, Purification and Formulation

1.4.1 Production and Characterization.

pET15-MOMP transformed BL21(DE3) bacteria were cultured into LB medium (Gibco BRL) supplemented with 200 μg/ml ampicilline (Sigma). Expression was induced by adding 1 mM IPTG when the culture optical density measured at 600 nm has reached 0.6 to 0.8. Cells are harvested 3 h after induction, washed 3 times with PBS and lysed in sample buffer containing 2% SDS and 5% mercaptoethanol. Samples were heated for 3 min at 95° C. and total proteins were separated by 12% SDS-PAGE using molecular weight markers separated on the same gel (Gibco BRL). For immunoblotting, the 12% SD-PAGE separated proteins were transferred onto nitrocellulose and detected using mAbs L2 I-45 and L2 I-10 kindly provided by Dr. H. Caldwell and a goat anti-MOMP antiserum (Chemicon). The cellular location of the rMOMP was determined by cell fractionation as described in Maniatis et al. (Sambrook, et al., Molecular Cloning. A Laboratory Manual. Second Edition. Cold Spring Harbor Laboratory Press, 1989); pellet and supernatants were resuspended or adjusted in sample buffer and analysed like the total cell lysate.

1.4.2 Purification.

A 100 ml volume of 3 h IPTG induced culture was lysed with lysozyme and deoxycholate as described previously by Marston et al. (Marston, et al., DNA Cloning: A Practical Approach, Vol. 3, pg. 59. Ed. D. M. Glover, IRL Press, Oxford). The cell lysate was centrifugated (12 000 g for 15 min.), the pellet was resuspended in 2 ml of SDS PAGE sample buffer containing 2% SDS but no mercaptoethanol and boiled for 3 min. The lysate was centrifugated (12 000 g for 15 min.), the pellet was discarded and the supernatant adjusted to 20 mM Tris-HCl pH 7.9, 0.5% SDS, 500 mM NaCl, 5 mM imidazole in final concentration. The sample was then loaded onto a chromatography column containing 2 ml His.Bind resin (Novagen); the ion metal affinity chromatography was then achieved according to the manufacturer's procedure. Identity and purity of the eluted product was estimated by SDS-PAGE under reducing conditions followed by Coomassie blue staining or immunoblotting (see above). Protein concentration was determined by the Lowry's assay. rMOMP containing fractions were pooled and dialysed overnight against PBS using Slide A Lyser cassettes (Pierce).

Formulation of the Antigen.

Three formulations were tested:
1) MOMP+QS21 3D-MPL
2) MOMP+SB62 (oil-in-water emulsion comprising squalene, alpha-tocopherol and tween 80)
3) MOMP, SB62, QS21 3D-MPL

The designation SB62 stands for SB's oil-in-water emulsion produced using methods as described in WO 95/17210. This was carried out according to the procedure described in WO 95/17210 and/or WO94/00153. Briefly purified and dialysed rMOMP was diluted in PBS to 25 μg/ml (200 μl) for injection with 5 μg 3D-MPL+10 μg QS21 or to 50 μg/ml (100 μl) for mixing with 100 μL oil in water emulsion referred to as SB62 with or without 5 μg 3D-MPL/10 μg QS21. For each of the formulations the vaccines were prepared as follow:
1) 3D-MPL/QS21 formulated by adding 3D-MPL (as 100 nm particles) to MOMP antigen, followed by buffer and then QS21.
2) 3D-MPL/QS21/SB62 formulated by adding antigen to buffer followed by SB62 followed by 3D-MPL as 100 nm particle followed by QS21. In this formulation it is believed that the antigen is out (i.e. outside the emulsion droplet), the 3D-MPL is out and most of the QS21 is out.
3) SB62 formulated by adding SB62 to antigen in buffer. Antigen is out.

A 1.5 Vaccination in the Mice Model of Salpingitis, Fertility and Serological Follow-Up

Groups of ten female C3H/HeOuJ were subcutaneously immunized at the basis of the tail with 2×5 μg rMOMP in 200 μl of the different formulations at weeks 0 and 2; the control group was sham-immunized following the same schedule with the emulsion containing 5 μg 3D-MPL and 10 μg QS21. Inoculation was carried out at week 6 following the protocol described previously by Tuffrey et al. (Tuffrey, et al., Br. J. Exp. Path., 67:605-616, 1986). Briefly, mice were given 2.5 mg progesterone subcutaneously (Depo-Provera, Upjohn) 7 days before challenge which was performed by bilateral intrauterine inoculation with 5 10⁵ IFU Chlamydia trachomatis NI1 in 100 μl SPG or 100 μl of a Mc Coy cell extract.

At week 10, treated mice were caged with male for 3 months for fertility assessment (1 male for 2 females, with weekly rotation of the male within each group). The parameters that were calculated over the breeding period were the mean number of newborn mice per group (M) and the average litter size (N).

Blood was taken at weeks 6 and the sera were analysed for rMOMP-specific antibodies, serovar F strain NI1 chlamydial inclusions recognition and neutralization of heterologous (NI1) in vitro infection. Vaginal washes were collected at week 6 by pipeting 50 μl of PBS into and out of the vagina several times and analysed for rMOMP specific secretory IgA antibodies.

A 1.6 Serological Analysis

1.6.1 Determination of Anti-rMOMP Antibodies.

The rMOMP-specific IgG or IgA titers were determined using the rMOMP as antigen in an enzyme-linked immunosorbent assay (ELISA). The plates (Maxisorp, Nunc) were coated overnight at 4° C. with a 5 μg/ml solution of antigen in 10 mM carbonate/bicarbonate buffer, pH 9.6 buffer, washed with 0.1% Tween 20 PBS (washing buffer) and blocked for 1 h at 37° C. with PBS 3% BSA (Sigma). Test sera were serially diluted in washing buffer containing 0.5% BSA (incubation buffer) for 1 h at 37° C. The plates were washed and incubated for 1 h at 37° C. with a horseradish peroxydase-congugated goat anti-murine IgG or IgA (Sigma). After washing, the substrate orthophenylen-diamine (Sigma) was added at room temperature for 20 min; the reaction was stopped by addition of 2M H₂SO₄ and the absorbance at 492 nm was measured on a Labsystems Multiskan. The anti-rMOMP IgG or IgA titer was expressed as the reciprocal of the serum sample dilution giving a midpoint absorbance value.

For each serum sample, the rMOMP-specific IgG response was dissected into rMOMP-specific IgG2a, IgG2b and IgG1 ratios in a direct ELISA as described above with some modifications. Test sera were incubated in triplicate, the plates were washed and biotinylated goat anti-murine IgG2a, IgG2b or IgG1 (Amersham) diluted in incubation buffer were added to each lane of the triplicate. After 1 h at 37° C., the plates were washed and incubated for 1 h with a streptavidine horseradish peroxydase complex (Amersham). Revelation and titer determination were carried out as described above. The prevalence of each of the 3 IgG subtypes expressed in percent was calculated as the ratio between this IgG subtype titer and the total of the titers determined for the 3 subtypes.

1.6.2 Heterotypic Detection of Chlamydial Inclusions.

Mac Coy cells were cultured in sterile flat-bottom 96-well microplates (Nunc) and confluent monolayers were infected with approximately 5 10⁴ IFU of Chlamydia trachomatis serovar F strain NI1. 24 h post-infection, the cells were washed with PBS and fixed 10 min with methanol. Washing was repeated and 100 μl of the serum samples diluted 1/100 with PBS were incubated for 1 h at 37° C. The plates were washed and treated with horseradish peroxydase-conjugated goat anti-mouse IgG (Sigma) for 1 h at 37° C. After washing with PBS, the antibody binding was visualized by addition of diaminobenzidine tetrahydrochloride (DAB, Sigma). The presence of anti-rMOMP IgG reveled NI1 inclusions was assessed using an inverted optical microscope.

1.6.3 Heterotypic in Vitro Neutralizing Activity.

The complement independent in vitro neutralization assay was performed as described by Su et al. (Su, et al., Vaccine, 11:1159-1166, 1993) with some modifications. Briefly 50 μl twofold SPG dilution of decomplemented individual mouse sera were added to 10⁵ IFU of serovar F strain NI1 diluted in 50 μl SPG. The mix was incubated 30 min at 37° C. (5% CO2) and then the 100 μl were inoculated onto HBSS (Gibco BRL) washed Syrian Hamster Kidney cells (HaK, ATCC, Rockville, Md.) and incubated for 2 h at 37° C. (5% CO2). Then, the inocula were removed, the cells were washed with HBSS and MEM containing 10% FCS, 50 μg/ml gentamycin and 0.5 μg/ml cycloheximide was added. After 24 h incubation at 37° C., cells were fixed and inclusions were immunochemically detected as described above using a commercial goat anti-MOMP antisera (Chemicon) and an alkaline phosphatase conjugated rabbit anti-goat (Sigma). 5-brom-4-chloro-3-indolyl phosphate/nitro blue tetrazolium (BCIP/NBT, Sigma) was used as the substrate for the enzymatic reaction. IFU were quantitated by counting 5 fields at a magnification of 200× using an inverted microscope. The mean IFU number per field obtained with the sample sera was expressed as percentage reduction of the mean IFU number obtained with negative control mixture which contained serum from naive mice. The neutralization titers (NT 50) were calculated as the reciprocal of the serum sample dilution giving 50% reduction of the infectivity.

A 1.7 Results

1.7.1 Recombinant Antigen Expression and Characterization

A PCR amplified DNA fragment containing the nucleotide sequence coding for mature MOMP serovar L2 was inserted in the right reading frame and orientation into the pET15 expression vector; the nucleotide sequence of the chlamydial protein and the fusion joint with the polynucleotide stretch encoding the 5′-terminal His-Tag peptide were as predicted by the design of the cloning strategy. After cell fractionation, the expression product was located in the insoluble fraction of E. coli which suggests that it was expressed in the form of insoluble inclusion bodies. The recombinant MOMP containing pellet was solubilized in 2% SDS buffer and run onto a ion metal affinity chromatography column in which immobilized nickel ions were used to chelate histidine residues beared by the His.tag peptide fused with the recombinant MOMP. The purified protein which has been washed and eluted in buffers devoid of SDS displayed the predicted molecular weight and was immunoreactive with anti-MOMP monoclonal and polyclonal antibodies as shown by SDS-PAGE and Western blot analysis respectively. After dialysis, the rMOMP concentration was situated between 500 μg/ml and 1 mg/ml and purity of the recombinant product was estimated at 90%.

1.7.2 Effect of Immunization with Adjuvanted rMOMP on Mouse Serological Response and Fertility After Heterotypic Challenge

The results of the experiment designed to evaluate the prophylactic potential of differently adjuvanted recombinant MOMP are presented as outlined in Table 1.

TABLE 1
Serological and fertility analysis in immunized and control mice monitored over a 12 weeks period.

| Group No. | Mean titer of | | Serovar F | | No. of mice | N (no. of newborn | |
|---|---|---|---|---|---|---|---|
| Immunization/ | rMOMP - specific | Mean ratio of | inclusions | Neutralisation | litter:no. | mice in group:no. of | M (Average |
| infection schedule | total IgG | IgGI:IgG2a:gG2b | detection^{a} | (NT 50)^{b} | of mice in group | mice in group) | litter size) |
| G1 | <100 | ND | No | <20 | 8/8 | 10.9 | 4.7 |
| Untreated | | | | | | | |
| G2 | <100 | ND | No | <20 | 3/10 | 1.3 | 2.3 |
| 3D-MPL + QS21 + SB62 | | | | | | | |
| Infected | | | | | | | |
| G3 | 34800 | 44.6:42.6:12.8 | Yes | <20 | 7/8 | 8.7 | 3.7 |
| MOMP + 3D- | | | | | | | |
| MPL + QS21 + SB62 | | | | | | | |
| Mock-infected | | | | | | | |
| G4 | 43000 | 58.3:35:12.7 | Yes | <20 | 7/10 | 3.4 | 2.8 |
| MOMP + 3D- | | | | | | | |
| MPL + QS21 + SB62 | | | | | | | |
| Infected | | | | | | | |
| G5 | 113000 | 28.9:66.6:4.5 | Yes | <20 | 7/10 | 4.5 | 3.8 |
| MOMP + 3D- | | | | | | | |
| MPL + QS21 | | | | | | | |
| Infected | | | | | | | |
| G6 | 43000 | 83:14.6:2.4 | Yes | <20 | 2/10 | 0.6 | 3 |
| MOMP + SB62 | | | | | | | |
| Infected | | | | | | | |

Groups 4 and 5 were subcutaneously immunized with rMOMP adjuvanted with 5 μg 3D-MPL and 10 μg QS21; in group 4, the adjuvanted recombinant protein was prepared in the SB62 emulsion containing squalene and alpha tocopherol as the oil phase and Tween 80 as the surfactant. Group 6 was subcutaneously immunized with rMOMP combined with the same SB62 emulsion as group 4 but without immunostimulants. Three control groups were also designed. A group of non treated animals (group 1), a group of sham-immunized mice using both immunostimulant combined with SB62 emulsion (group 2) and group 3 which was immunized like group 4 but was devoted to mock-infection. group 2, 4, 5 and 6 were challenged with the heterotypic Chlamydia trachomatis strain NI1.

1.7.3 Effect of Immunization on the Serological Response.

In order to evaluate the immunogenicity of the different preparation, IgG titers were measured by ELISA on sera drawn 4 weeks after the second dose of vaccine (day of the challenge) and the arithmetic mean titers (AMT) were calculated for each group. Immunisation with two injections of 5 μg of rMOMP led to the appearance of high level of anti-rMOMP IgG. As shown in Table 1, AMTs were virtually the same for groups 4 and 6 but combination of emulsion and immunostimulants resulted in a twofold increase of the rMOMP specific IgG mean titer. Animals sham-immunized with adjuvants only (group 2) had no significant antibody titers against the chlamydial recombinant antigen. In any groups, no specific rMOMP secretory IgA were detected in the vaginal washes collected just before challenge.

As shown in Table 1, a significant difference in the IgG subclasses profile was observed between the immunostimulants containing groups (4 and 5) and group 6 which was immunized with SB62 emulsified rMOMP. The utilization of 3D-MPL+QS21 was shown to significantly enhance the relative level of IgG2a and decrease the relative level of IgG1; the maximum effect of this phenomenon was reached with the non-emulsified preparation.

To ascertain whether the rMOMP specific IgG were able to cross-react with Chlamydia of the heterotypic infecting strain, sera were diluted 1:100 and individually tested in an immuno-enzymatic assay for their ability to recognize chlamydial inclusion on methanol fixed infected cells. All the mouse sera from each immunized group were shown to contain IgG reacting with the Chlamydia trachomatis serovar F strain NI1 utilized for the challenge. Therefore, they were all tested for in vitro complement-independent neutralizing activity against this strain using sera from sham-immunized mice as negative controls. Results were inconsistent in comparison to those obtained by ELISA and immunoenzymatic-assay since none of the immune sera was able to significantly reduce the chlamydial infectivity.

1.7.4 Effect of Heterotypic Immunization on the Fertility After Challenge.

Eight weeks after the last immunization (or sham-immunization) with rMOMP and 4 weeks after intrauterine infection (or mock-infection), mice were mated with male for 2 months. The outcome of the challenge with the heterologus strain NI1 on C3H/He0uJ mice fertility was measured through the following parameters: the mean number of newborn per group (N) and the average litter size (M). Compared to untreated mice, fertility of Chlamydia inoculated mice in group 2 (sham-immunized) were significantly altered; this result confirmed the validity of the animal model in this particular case. Animals immunized and mock-infected (group 3) presented reduced parameters of fertility compared to untreated ones; this group designed to take non-pathological alteration of the animal fertility into consideration was used as control to evaluate the prophylactic potential of the rMOMP formulations. As shown in Table 1 significant differences in the fertility levels were observed between the immunostimulants co-vaccinated groups (groups 4 and 5) and group 6 which was immunized with SB62 emulsified rMOMP. Group 5 displayed the best results with 7 out to 10 mice giving birth to litters and maximal fertility parameters; the N value reached 50% of the value attributed to the control group 3 and the M value was comparable to those calculated for the same control group. On the contrary, the fertility parameters displayed by group 6 lacking immunostimulants is comparable to those obtained in the infection control group 2. rMOMP immunization combining immunostimulants and the SB62 emulsion also resulted in protection against infertility but the utilization of the SB62 emulsion seemed to partially decrease the protection rates. Thus, the utilization of 3D-MPL plus QS21 was shown to offer a partial protection against upper genital tract chlamydial infection and the maximum effect of this phenomenon was reached with the non-emulsified preparation.

A 1.8 Conclusion

Our results show that parenteral immunization with 3D-MPL+QS21 adjuvanted rMOMP partially prevent infertility caused by an heterologus chlamydial infection of the mouse genital tract; on the contrary, injection of SB62 emulsified rMOMP without both immunostimulants do not induce any protection. On the other hand, all the preparations elicited strong and relatively homogeneous MOMP specific total IgG response in the sera of immunized animals; those antibodies were able to cross-react with methanol fixed chlamydial inclusions of the heterotypic infecting strain but unable to reduce the chlamydial infectivity in vitro. Just before challenge, rMOMP specific IgA were not detectable in the vaginal secretion which is consistent with the parenteral antigen administration method. Thus, a comparison of total specific IgG titers or neutralization titers with the outcome of the pathology for all immunized groups revealed no significant correlation for any of these comparison.

Results from the present investigation demonstrate that a recombinant MOMP combine with 3D-MPL+QS21 immunostimulants is capable of eliciting immune protection against infertility caused by Chlamydia trachomatis.

A 2. Second Series of Experiments with Various MOMP Adjuvant Formulations

A 2.1 Material and Methods were Tested

Chlamydial and mouse strains were identical to those utilised in the experiment described in Example 1.

A 2.2 MOMP Production

The production and the use of the DNA construct pET15-MOMP for antigen production are described in Example 1. The antigen purification protocol was modified in order to produce larger quantities of endotoxin-free antigen. Briefly, 10 ml His. Bind resin (Novagen) were washed with 25 volumes of 2% SDS, 6M urea in water before performing the purification step according to the manufacturer while 250 ml of induced lysate pelleted by centrifugation was washed with 4M urea, 2M NaCl followed by 2% Zwittergen (Calbiochem) before solubilisation in Tris-HCl pH 7.9, 0.5% SDS, 500 mM NaCl, 5 mM imidazole. The antigen was eluted in Tris-HCl pH 7.9, 100 mM imidazole, extensively dialysed against 5 mM Tris-HCl pH 7.4 and filtered onto a 0.22 μm sterilising filter until (Millipore). A limulus amaoebocyte lysate test (Coatest, Chromogenix) was then used to assess the LPS content.

A 2.3 Formulation

The antigen was formulated as described in A.1.4 except that the quantity of 3D-MPL per dose was adjusted to 10 μg; a group utilising modified QS21 as described in copending UK application 9513107.4 and WO 96/33739 (referred as DQ) was also tested and added to the vaccine at the rate of 10 μg a dose. In more detail the vaccine was formulated by adding antigen to buffer. 3D-MPL as 100 nm particles was then added. In separate tube, QS21 was mixed with small unilamellar liposomes composed of dioleoylphosphatidylcholine (DOPC) and cholesterol (DOPC:cholesterol=4:1 w/w) so that the QS21 to cholesterol ratio is 1:5. (Under these conditions all the QS21 is incorporated into the liposomal membrane). The QS21/SUV mix (called DQ) is then added to the antigen/3D-MPL mix. In this formulation the antigen is out, the 3D-MPL is out, the QS21 is in liposomes.

A 2.4 Vaccination in the Mice Model of Salpingitis, Fertility, Immunological and Histological Follow-Up.

Immunisation, experimental infection and sampling were scheduled as described above except that the negative control group was sham-immunised with the emulsion containing 10 μg 3D-MPL and 10 μg QS21 and that an extra group immunised with the antigen combined with 3D-MPL+DQ was added. Groups were composed of 15 mice: 10 of them were mated over a 8 week period while 5 were sacrificed 2 weeks post challenge for histopathological and immunocytochemical analysis. The parameters used for estimating group's fertility are: F (number of mice which littered one time or more divided by the total number of mice), M (number of newborn mice (dead or alive) divided by the number of litters) and N (number of newborn mice (dead or alive) divided by the total number of mice).

Sera and vaginal washes were analysed for rMOMP-specific antibodies by ELISA, sera were also examined for serovar F strain NI1 chlamydial inclusions recognition and neutralisation of heterologus (NI1) in vitro infection. All the techniques are described supra.

Upper half genital tract (ovary, oviduct and top of the uterine horn) were embedded in OCT compound (Tissue-TEK, Miles), snap frozen and frozen sections (10 μm) were mounted on glass slides (Superforst, Menzel-glaser). Sections were air-dried, fixed in acetone for 5 minutes and then stored at −70° C. For histopathological analysis, water rehydrated sections were stained with haematoxylin (H) and eosin (E). For immunocytochemical staining, sections were rehydrated in PBS, incubated for 60 minutes with 2 μg of biotinylated rat anti-mouse CD4 or CD8 mAb (Serotec) in 100 μl PBS, washed 2 times with PBS and reincubated 30 minutes with a 1/2000 dilution of HRP-streptavidin (Zymed). After washing, colour was developed with a liquid DAB kit (Zymed), countersigned with haematoxylin and permanently mounted in acrytol (Surgipath).

A 2.5 Interferon Gamma Assay

Mice were subcutaneously injected into the basis of the tail with 200 μl of formulation on weeks 0 and 2; the control group was sham-immunised with 10 μg 3D-MPL and 10 μg QS21 combined to the emulsion. Animals were bled for serological analysis and then sacrificed on week 4, spleens were aseptically removed, pooled and single cell suspension were prepared for restimulation with 1 μg/ml rMOMP or with 4/ml Con A (Boehringer Mannheim) as a control. Therefore cultures were set up in flat bottom 24-well culture plates using 10⁶ responder cells per ml of RPMI 1640 with 10% foetal calf serum (Gibco-BRL). Supernatants harvested at 96 hours post restimulation were assayed for IFN-gamma using a commercial ELISA kit (Cytoscreen, Biosource).

A 2.6 Results

2.6.1 Antigen

After dialysis, the rMOMP concentration was estimated around 2 mg/ml and endotoxins contamination was below 0.05 EU/μg rMOMP/

2.6.2 Effect of Immunisation with Adjuvanted rMOMP on the Mice Humoral Immune Response, on Their Fertility After Heterotypic Challenge and on the Inflammatory Infiltrate Resulting from Infection.

The results of the experiment designed to evaluate the prophylactic potential of differently adjuvanted rMOMP are presented as outlined in Table 2.

TABLE 2

| Group No. | rMOMP - | | | N: mean | M: |
|---|---|---|---|---|---|
| Immunization/ | specific | Isotype | F: proportion | nber of | mean |
| infection | IgG | ratios | of fertile mice | newborn | litter |
| schedule | GMT | IgG2a;IgG1;IgG2b | (%) | per mouse | size |
| G1 (Negative | <100 | ND | 1/8 | 0.8 | 4 |
| Ctrl) - | | | (12.5) | | |
| (SC) | | | | | |
| 3D- | | | | | |
| MPL + QS21 + SB62/ | | | | | |
| Infected | | | | | |
| G2 (Positive | 23900 | 44;52;4 | 9/10 | 6.2 | 3.6 |
| Ctrl) | | | (90) | | |
| rMOMP L2 (sc) | | | | | |
| 3D- | | | | | |
| MPL + QS21 + SB62/ | | | | | |
| Sham-infected | | | | | |
| G3 | 32820 | 49;47;4 | 4/9 | 1.7 | 3 |
| rMOMP L2 (Sc) | | | (44) | | |
| 3D- | | | | | |
| MPL + QS21/Infected | | | | | |
| G4 | 41415 | 14;83;3 | 3/10 | 1.9 | 4.75 |
| rMOMP L2 (Sc) | | | (30) | | |
| SB62/Infected | | | | | |
| G5 | 30830 | 32;65;3 | 6/8 | 4.8 | 3.3 |
| rMOMP L2 (sc) | | | (75) | | |
| DQ + 3D-MPL/ | | | | | |
| Infected | | | | | |

Sc: subcutaneous route
GMT: geometric mean titer
ND: not done

2.6.3 Effect of Immunisation on the Humoral Response.

Humoral response after vaccination was assessed in sera and vaginal washes collected on the day of the challenge. All the formulations of the antigen gave similar anti-rMOMP IgG geometric mean titers (GMT) of around 30,000. Animals sham-immunised with adjuvants only had no significant antibody titers against the chlamydial recombinant antigen. In any groups, no rMOMP-specific secretory IgA were detected in the vaginal washes collected just before challenge. Isotyping the rMOMP-specific IgG response revealed that the presence of 3D-MPL and QS21 or DQ enhanced the relative level of IgG2a when compared to the response evoked by MOMP in emulsion alone. All the immune sera were shown to contain IgG reacting with methanol-fixed inclusions of the serovar FC trachomatis strain NI1 utilised for the challenge.

2.6.4 Effect of Heterotypic Immunisation on the Fertility After Challenge.

The outcome of the challenge with the heterologus strain NI1 on mice fertility was measured through F, N and M parameters defined in the experimental procedures. For each group, values of those parameters calculated over the duration of the mating period are presented at Table 2. In opposition to the sham-infected group, infection of the sham-immunised group led to nearly complete infertility, indicating that the observed infertility is induced by C. trachomatis and not by the manipulation of the animals. Among the groups immunised with rMOMP adjuvanted with both immunostimulants, 3D-MPL+DQ formulation of the antigen led to partial protection: in this group the values of the F and N parameters reach around 80% of those recorded in the sham-infected group (positive control). On the contrary, immunisation prior to challenge with rMOMP formulated in the emulsion led to low values of the F and N fertility parameters.

2.6.5 Histopathological Changes After Challenge

In the histopathological counterpart of the fertility experiment, classical HE coloration performed on tissue sections revealed no markedly difference in the inflammation scores of the oviducts and the ovaries between sham-immunised and vaccinated groups. However, when looking at the frequence of T-cell subsets by immunocytochemical straining, CD4 positive T-cells were only detected in the 3D-MPL+DQ vaccinated group (4 of 4 mice), whereas CD8 positive T-cells were detected in immunised as well sham-immunised groups (Table 3 below). Thus, in this experiment, CD4 positive infiltrating T-cells were only found in the 3D-MPL+DQ vaccinated group which was the only group to be protected in the fertility counterpart of the experiment.

TABLE 3

| Group No. | | | | | |
|---|---|---|---|---|---|
| Immunization/ | | CD8 Positive | | CD4 Positive | |
| infection | Mouse | T-cell score | | T-cell score | |
| schedule | No | Oviduct | Ovary | Oviduct | Ovary |
| G1 (Negative | 1 | +/++ | ++ | − | − |
| Ctrl) - | 2 | +/++ | +/++ | − | − |
| (SC) | 3 | ND | ND | ND | ND |
| 3D- | 4 | ND | ND | ND | ND |
| MPL + QS21 + SB62/ | | | | | |
| Infected | | | | | |
| G2 (Positive | 1 | − | − | − | − |
| Ctrl) | 2 | − | − | − | − |
| rMOMP L2 (sc) | 3 | − | − | − | − |
| 3D- | 4 | − | − | − | − |
| MPL + QS21 + SB62/ | | | | | |
| Sham-infected | | | | | |
| G3 | 1 | + | + | − | − |
| rMOMP L2 (sc) | 2 | +/++ | + | − | − |
| 3D-MPL + QS21/ | 3 | +/++ | +/− | − | − |
| Infected | 4 | +/− | − | − | − |
| G4 | 1 | +/− | +/− | ND | ND |
| rMOMP L2 (sc) | 2 | +/++ | + | − | − |
| SB62/ | 3 | +/++ | + | − | − |
| Infected | 4 | +/++ | + | − | − |
| G5 | 1 | +/++ | ++ | +/− | +/++ |
| rMOMP L2 (sc) | 2 | ++ | + | − | +/− |
| DQ + 3D-MPL/ | 3 | +/++ | ++ | − | + |
| Infected | 4 | ++ | + | +/− | +/++ |

sc: subcutaneous route
ND: not done
CD4 or CD8 positive T-cell scores were graded from no cell (−) to maximal (+++) infiltration of the considered cell type

2.6.6 Effect of the Formulation on IFN-Gamma Secretion Upon in Vitro Restimulation

The cellular activation induced by the antigen formulations (Table 4 below) was analysed in a separate experiment. On one hand, spleen cells isolated from animals vaccinated with rMOMP combined with 3D-MPL+QS21 or 3D-MPL+DQ and in vitro restimulated with the antigen displayed IFN-gamma concentrations in their culture supernatants which are comparable to those stimulated during the same period with 4 μg/ml of concanavaline A. On the other hand, cells isolated from animals sham-vaccinated or vaccinated with rMOMP devoid of immunostimulants did not produce detectable levels of IFN-gamma while their counterpart co-cultured with ConA were all positive for that cytokine. Serological analysis performed on pools of sera from each group revealed that IFN-gamma secretion was associated with an enhancement of the antigen-specific IgG2a ratio.

TABLE 4

| | rMOMP - | | γ-IFN (pg/ml) | γ-IFN (pg/ml) |
|---|---|---|---|---|
| Group No. | specific | | Restimulation | Restimulation |
| Immunization/ | IgG | Isotype ratios | with ConA | with rMOMP |
| schedule | GMT | IgG2a;IgG2b;IgG1 | (4 μg/ml) | (1 μg/ml) |
| G1 (Negative Ctrl) - | ND | ND | 555 | <25 |
| (SC) | | | | |
| 3D- | | | | |
| MPL + QS21 + SB62 | | | | |
| G2 | 18000 | 8.8;3.5;87.7 | 353 | <25 |
| rMOMP L2 (sc) | | | | |
| SB62 | | | | |
| G3 | 49000 | 70;6.8;23.2 | 397 | 503 |
| rMOMP L2 (sc) | | | | |
| 3D-MPL + QS21 | | | | |
| G4 | 42000 | 57.5;6;36.5 | 461 | 258 |
| rMOMP L2 (sc) | | | | |
| DQ + 3D-MPL | | | | |

sc: subcutaneous route
GMT: geometric mean titer
ND: not done

A 3 Conclusion

We have shown that immunisation with a vaccine comprising 3D-MPL and QS21 or DQ and MOMP from serovar L2 is effective in conferring protection against infertility resulting from heterologous Chlamydial infection (Lucero, et al., Infect. Immun., 50:595-597, 1985). Indeed, data from the challenge trial and the IFN-gamma detection assay suggest that, in mouse, the combination of the two adjuvants 3D-MPL and QS21 or DQ with a recombinant MOMP induces an antigen-specific Th1-like immune response determined by IFN-gamma secretion and elevated IgG2a ratios, which can result in protection against infertility resulting from chlamydial infection.

B. Set of Experiments Performed with CT and mLT as Adjuvant

B 1. Material and Methods

B 1.1. Purified rMOMP Production and Formulation

The obtention and the use of the DNA construct pET15-MOMP for antigen production are described in the U.K. patent GB 9506863.1 published as PCT No. 96/31236. Purification of the protein was carried out under denaturing conditions using His.Bind resin (Novagen) as disclosed by the same patent; the LPS and the protein concentrations were measured in the final product using a Limulus amoebocyte lysate test (Coatest, Chromogenix) and the BCA method (BCA kit, Pierce) respectively. Doses of vaccine devoted to intra-nasal immunisation were prepared by mixing 10 μg mLT (obtained from SmithKline Beecham Biologicals) or CT (Swiss Serum, Bern) with 10 μg of rMOMP serovar F (rMOMPF) or L2 (rMOMPL2) in a final volume of 20 μl PBS.

B 1.2.Vaccination in the Mouse Model of Salpingitis, Fertility, Sampling and Immunological Follow-Up.

Groups of ten female C3H mice (6 weeks, Iffa Credo) were immunised at week 0 and 2 by intra-nasal administration of 20 μl of the vaccine formulation containing CT or mLT under Hypnorm (Janssen-Cilag) and Dormicum (Roche) anesthesia. The experimental challenge was carried out as following: at week 5, mice were given 2.5 mg progesterone intra peritoneally (Depo-Provera, Upjohn) and at week 6, they were infected by bilateral intrauterine inoculation with 5×10⁵ inclusion forming units (IFU) C. trachomatis NI1 (serovar F) in 100 μl sucrose phosphate glutamate buffer (SPG) or with 100 μl of a Mc Coy cell extract for the fertility positive control group.

At week 10, treated mice were caged with males for 3 months for fertility assessment (1 male for 2 females per cage with weekly rotation of the males within each group); the parameters used for estimating group's fertility were: F (number of mice which littered one time or more divided by the total number of mice), M (number of newborn mice (dead or alive) divided by the number of litters) and N (number of newborn mice (dead or alive) divided by the total number of mice).

Determination of the MOMP-Specific Humoral Response

Sampling and quantification of antibody (Ab) responses by ELISA were performed on individual animals as disclosed in the patent GB 9506863.1 supra with some modifications. Vaginal secretions were collected at weekly intervals from week 3 until week 7 by repeated flushing and aspiration of 50 μl PBS, diluted 1:4 in PBS containing 0.5% BSA and 0.1% Tween 20 and analyzed for rMOMP-specific secretory IgA or IgG antibodies. Since the concentration of specific Ab can be affected by variations in fluid recovery during the lavage, total IgA were also quantified but only in the first experiment. Since we detected little or no variation in total Ab level (not shown) between analyzed mice, subsequent vaginal washing were devoted to MOMP-specific IgA analysis only. In order to assess the effectiveness of the intra-nasal immunisation, CT-specific IgA and IgG were also determined in the samples from the first experiment. Titers were determined arbitrarly as the reciprocal of the sample dilution corresponding to an optical density of 1 at 492 nm and mice that displayed at least once a titer higher or equivalent to 4 were considered to be positive for antigen-specific IgA.

Blood samples were collected at week 6 (week of the challenge) and sera were analysed for the presence of rMOMP-specific IgG. In the first experiment, CT-specific IgG were also determined in the serum in order to make sure of the effectiveness of the intra-nasal immunisation; therefore, microtiter plates were precoated with 0.5 μg of CT (Swiss Serum, Bern) per well and then processed as described in patent GB 9506863.1.

Determination of the MOMP-Specific Cellular Response

Two groups of five female C3H mice (6 weeks, Iffa Credo) were immunised at week 0 and 2 by intra-nasal administration of 20 μl of the vaccine formulation containing mLT under Hypnorm (Janssen-Cilag) and Dormicum (Roche) anesthesia; negative control groups were sham-immunised with the mLT only following the same procedure. Animals from group 1 and 2, and those from corresponding controls were bled for serological analysis and sacrificed on day 9 and 19 after the second boost respectively; spleens were aseptically removed, pooled and single cell suspension were prepared for restimulation with 1 μg/ml rMOMP serovar L2 or with 4 μg/ml Concanavalin A (Boerhinger Mannheim) as a positive control; unrestimulated cultures were used as negative control of the cellular activation.

For the measurement of cell proliferation, triplicates cultures were set up in round bottom 96-well culture plates using 5×10⁴ responder cells per well in 200 μl of RPMI 1640 with 10% foetal calf serum (FCS, Gibco-BRL); after 72 hours of incubation at 37° C. in 7% CO₂, supernatants (SN) were recolted while cells were pulsed for 18 h with 1 μCi of tritiated thymidine (Amersham) per well, harvested onto glass-fiber (Skatron), air dried and counted for beta emission by standard liquid scintillation. The stimulation index (SI) which is the mean of antigen or ConA-stimulated T-cell uptake of tritiated thymidine for triplicate wells divided by the mean of unstimulated T-cell uptake for triplicate wells, was calculated for each group.

IFN-gamma was determined in culture SN using a commercial ELISA kit (Duoset, Genzyme). For cells obtained at day 9 after boosting, 72 h culture SN of the lymphoproliferative assay pooled per triplicate were used while for those obtained at day 19, 48 h culture SN from 24-well plates especially established for that purpose (5×10⁶ cells per ml of RPMI 1640 containing 10% FCS) were used.

B2. Results

Evidence that mucosal immunisation with rMOMP combined with CT or mLT can afford protection against infertility caused by Chlamydial challenge is given by the first two experiments described below. As these experiments were primarily designed for evaluation of systemic immunisation (not shown), the negative and positive control groups were subcutaneously treated with adjuvants other than CT or mLT; rMOMP-naive animals (negative control groups) were infected to ascertain the effect of the challenge on the fertility while rMOMP-immunised animals (positive control groups) were sham-infected in order to take into consideration the alteration of the fertility that could result from the manipulation of the animals during intrauterine inoculation.

A third experiment was set up in order to characterize the cellular activation evoked by rMOMP adjuvanted with mLT wherein the negative control group consisted in mice intra nasally sham-immunised with mLT alone.

B 2.1. Experiment 1

In the first experiment (Table 5 below), intra-nasal immunisation with rMOMPF+CT was evaluated for its protective effect against infertility caused by Chlamydial infection (homotypic challenge). Analysis of the humoral immune response just before challenge revealed that all the mice displayed CT-specific IgG in their serum and CT-specific IgG and IgA in their vaginal secretions, but no detectable rMOMP-specific IgG or IgA responses in the same prelevements, respectively. However, after challenge, this group displayed values of the F and N fertility parameters which reached 77 and 66%, respectively, of those of the positive control group, while the negative control group was nearly completely infertile (14% of the F and 13% of the N values recorded in the positive control group).

TABLE 5

| Group No. | rMOMP-specific | rMOMP-specific | | | |
|---|---|---|---|---|---|
| Immunisation/ | IgG geometric | IgA positive mice | F: proportion of | N: mean nber of | |
| infection schedule | mean titer (serum) | (vaginal washes) | fertile mice | newborn per mouse | M: mean litter size |
| G2 (NEGATIVE CTRL) - | <100 | ND | 1/8 | 0.8 | 4 |
| (sc) | | | | | |
| 3D-MPL + QS21 + SB62/ | | | | | |
| Infected | | | | | |
| G3 (POSITIVE CTRL) | 23900 | ND | 9/10 | 6.2 | 3.6 |
| rMOMP L2 (sc) 3D- | | | | | |
| MPL + QS21 + SB62/Sham- | | | | | |
| infected | | | | | |
| G8 rMOMP F (in) | <100 | 0/10 | 7/10 | 4.1 | 3.4 |
| CT/Infected | | | | | |

SC: subcutaneous
IN: intra-nasal

B 2.2. Experiment 2

In the second experiment (Tables 6 and 7 below), groups of mice were intra-nasally immunised either with rMOMPF combined with CT, or with rMOMPL2 combined with CT or mLT; in addition to the negative and positive control groups described above, a sham-immunised control group, intra-nasally treated with CT alone, was included in the experiment. As observed in the first experiment, intra-nasal administration of rMOMPF+CT did not induce any detectable humoral rMOMPF-specific response, neither in the sera collected just before challenge (IgG response), nor in the vaginal secretions collected weekly from boosting immunisation to challenge (IgA response). On the contrary, intra-nasal administration of rMOMPL2 combined with CT or mLT induced an antigen-specific humoral response in some of the animals: 1 and 3 out of 10 mice, respectively, were found to be IgG positive when analyzing sera collected just before challenge, while 5 and 7 out of 10 mice, respectively, were found to be IgA positive at least in one of the vaginal washes collected every weeks from boosting immunisation to challenge. Infection did not boost the MOMP-specific IgA response as shown by analysis performed one week after challenge.

TABLE 6

| | | | | IgA | IgA | | | | IgG |
|---|---|---|---|---|---|---|---|---|---|
| | Mouse | | | week 3 | week 4 | IgA | IgA | IgA | week 6 |
| Group | number | Formulation | Route | (vaginal | washes) | week 5 | week 6* | week 7** | (serum) |
| G1 | 1 to 10 | — | SC | ND | ND | ND | ND | ND | ND |
| | | DQ | | | | | | | |
| G2 | 1 to 10 | rMOMP L2 | SC | ND | ND | ND | ND | ND | 9000 |
| | | 3D- | | | | | | | (GMT) |
| | | MPL + DQ | | | | | | | |
| G3 | 1 | rMOMPL2 | IN | 8 | <4 | <4 | <4 | <4 | <100 |
| | | mLT | | | | | | | |
| | 2 | | | <4 | <4 | <4 | <4 | <4 | <100 |
| | 3 | | | 85 | >108 | 140 | 8 | >162 | 3400 |
| | 4 | | | 10 | 22 | 14 | 5 | 10 | 260 |
| | 5 | | | 17 | <4 | <4 | <4 | <4 | <100 |
| | 6 | | | <4 | <4 | 110 | <4 | 45 | <100 |
| | 7 | | | <4 | <4 | <4 | <4 | 13 | <100 |
| | 8 | | | <4 | <4 | <4 | <4 | <4 | <100 |
| | 9 | | | <4 | <4 | <4 | 9 | <4 | <100 |
| | 10 | | | 18 | 22 | 7 | <4 | <4 | 120 |
| G4 | 1 to 10 | CT | IN | ND | ND | ND | ND | ND | ND |
| G5 | 1 | rMOMPL2 | IN | <4 | <4 | 4 | <4 | <4 | <100 |
| | | CT | | | | | | | |
| | 2 | | | <4 | <4 | <4 | <4 | <4 | <100 |
| | 3 | | | 43 | 400 | 140 | <4 | 16 | 170 |
| | 4 | | | <4 | <4 | <4 | <4 | <4 | <100 |
| | 5 | | | <4 | <4 | <4 | <4 | <4 | <100 |
| | 6 | | | <4 | <4 | <4 | <4 | <4 | <100 |
| | 7 | | | 6 | 13 | 10 | <4 | <4 | <100 |
| | 8 | | | 5 | <4 | 5 | <4 | <4 | <100 |
| | 9 | | | <4 | 30 | <4 | <4 | <4 | <100 |
| | 10 | | | <4 | <4 | <4 | <4 | <4 | <100 |
| G6 | 1 to 10 | rMOMP F | IN | <4 for all 10 | <4 for all 10 | <4 | <4 | <4 | <100 for all 10 |
| | | CT | | | | | | | |

*Day of challenge
**Post challenge

TABLE 7

| | rMOMP | | | |
|---|---|---|---|---|
| | IgG | IgA positive | | N.mean |
| Group No. | Geometric | mice | | nber of |
| Immunisation/infection | mean titer | (vaginal | Fertile | newborn |
| schedule | (serum) | washes) | mice | per mouse |
| G1 (NEGATIVE | <100 | ND | 1/9 | 0.2 |
| CTRL) - | | | | |
| (sc/sc) | | | | |
| DQ 3D-MPL/DQ 3D- | | | | |
| MPL | | | | |
| Infected | | | | |
| G2 (POSITIVE CTRL) | 9000 | ND | 8/8 | 6 |
| rMOMP L2 (sc/sc) | | | | |
| DQ 3D-MPL/DQ 3D- | | | | |
| MPL | | | | |
| Sham-infected | | | | |
| G3 | 473 | 7/10 | 10/10 | 9.1 |
| rMOMP L2 (in/in) | (on the 3 | | | |
| mLT/mLT | positive | | | |
| Infected | mice only) | | | |
| G4 - | ND | ND | 4/10 | 2.1 |
| (in/in) | | | | |
| CT/CT | | | | |
| Infected | | | | |
| G5 | <100 | 5/10 | 6/8 | 3.5 |
| rMOMP L2 (in/in) | | | | |
| CT/CT | | | | |
| Infected | | | | |
| G6 | <100 | 0/10 | 5/8 | 4.9 |
| rMOMP (in/in) | | | | |
| CT/CT | | | | |
| Infected | | | | |

When compared with the positive control sham-infected group, fertility in the negative control group was nearly completely abolished, indicating the specific effect of the Chlamydial infection. Fertility of the mucosally treated groups revealed that immunisation with rMOMPF or rMOMPL2 combined with CT gave similar level of protection (63 or 75% respectively of the F, and 81 or 58% of the N values recorded in the positive control group). Immunisation with rMOMPL2 combined with mLT gave the best level of protection, with the F value identical and the N value higher (150%) than those recorded in the positive control group. Administration of CT alone also seemed to reduce the infertility level, but to a lesser extent than the rMOMP+CT formulations with 40% of the F and 35% of the N values recorded in the positive control group.

B 2.3. Experiment 3

The cellular activation induced by the antigen formulated with mLT was analysed through cell proliferation and IFN-gamma secretion upon antigen-specific restimulation.

When tested at day 9 and 19 days after the boost, spleen cells from groups immunised with the antigen developed strong specific proliferative immune response (38% and 108% of the positive control respectively) while those from control animals that were sham-immunised with mLT alone did not respond to in vitro restimulation (Tables 8 and 9 below).

TABLE 8
Cellular response analysed 9 days after boost immunization.

| Group N° | | | γ-IFN |
|---|---|---|---|
| Immunization | Mean cpm | Stimulation | (pg/ml) |
| schedule: | (5 10⁴ cells/well) - | Index - | 2.5 10⁵ C/ml - |
| formulation | ConA | ConA | ConA |
| (route) | rMOMP | rMOMP | rMOMP |
| G1 - | 897 | 1 | <20 |
| mLT | 35672 | 40 | 863 |
| (IN) | 2516 | 2 | 137 |
| (sham-imm) | | | |
| G2 | 516 | 1 | <20 |
| rMOMPL2 | 30002 | 58.1 | 610 |
| mLT | 11517 | 22.3 | 572 |
| (IN) | | | |

TABLE 9
Cellular response analysed 19 days after boost immunization.

| Group N° | | | γ-IFN |
|---|---|---|---|
| Immunization | Mean cpm | Stimulation | (pg/ml) |
| schedule: | (5 10⁴ cells/well) - | Index - | 5.0 10⁶ C/ml - |
| formulation | ConA | ConA | ConA |
| (route) | rMOMP | rMOMP | rMOMP |
| G1 - | 4379 | 1.0 | <20 |
| mLT | 20712 | 4.7 | 4348 |
| (IN) | 5890 | 1.3 | <20 |
| (sham-imm) | | | |
| G2 | 1481 | 1.0 | <20 |
| rMOMPL2 | 22234 | 15.0 | 5826 |
| mLT | 24166 | 16.3 | 1790 |
| (IN) | | | |

Spleen cells collected at both timepoints and restimulated with the antigen displayed IFN-gamma concentrations in their culture supernatants which were in the range of those restimulated during the same period with 4 μg/ml of Con A. On the other hand, cells isolated from sham-vaccinated animals and cultured with the antigen produced relatively low levels of IFN-gamma when compared with their counterpart cultured with ConA (Tables 8 and 9 above).

When looking at the humoral response, we were unable to detect nor rMOMP-specific IgG in pools and individual sera, neither rMOMP-specific IgA in pools and individual vaginal washings and that for prelevements made at both timepoints.

These data show that mucosal administration of rMOMP, when combined with CT or mLT, elicits protection (either homotypic or heterotypic) against infertility caused by a Chlamydial challenge. The fact that the protection cannot be correlated with local rMOMP-specific IgA argues for the existence of immune protective mechanism(s) different from a specific secretory antibody response. Results from the later experiment suggest that, in mouse, intra nasal administration of rMOMP combined with mLT induce a specific Th1 T cell immune response which could be responsible for the protection observed.

C. Second Set of Experiments Performed with QS21+3D-MPL Combination as Adjuvant

C1. Materials and Methods

C 1.1. Purified rMOMP Production and Formulation

Recombinant MOMPL2 was produced as described in section B 1.1. Formulation of the antigen with 3D-MPL+DQ was performed as described in section A2.3; formulation of the antigen with mLT was performed as described in section B1.1.

C 1.2.Vaccination in the Mouse Model of Salpingitis, Fertility, Sampling and Immunological Follow-Up.

The same procedure as the one described in section B1.2 was used. Mice were vaccinated at week 0 and 2. At week 0, the vaccine formulations were administered by the subcutaneous route; at week 2, vaccine formulations were administered by either subcutaneous, intranasal, or intrarectal routes (see table 10 for detailed schedule). Intrarectal immunisation was performed using a syringe and a feeder needle; 50 microliters of formulation per animal were placed in the rectum at 2 centimeters from the anal orifice.

C2. Results

When looking at the sera from the mice enrolled in this experiment, in which immunization routes and/or formulations were combined, we detected antigen-specific IgG responses in all the animals (Table 10 below). The vaginal IgA responses obtained after intranasal boosting differed from those generated by systemic boosting (no responder) and by intrarectal instillation: intranasal recall with the rMOMPL2 combined with 3D-MPL/DQ or mLT induced relatively high and sustained antibodies in 9 out of 10 lice in both groups while only 5 out of 10 mice were found IgA positive after intrarectal boosting (positive antigen-specific IgA response detected in at least one of the vaginal washes collected every week from the second immunization until challenge).

TABLE 10

| | RMOMP | IgA | | N.mean |
|---|---|---|---|---|
| | IgG | positive | | nber of |
| | Geometric | mice | | newborn |
| Group No. | Mean titer | (vaginal | Fertile | per |
| Immunisation/infection schedule | (serum) | washes) | mice | mouse |
| G1 (NEGATIVE CTRL) | <100 | 0/10 | 1/9 | 0.2 |
| 3D-MPL/DQ at week 0 (SC) | | | | |
| 3D-MPL/DQ at week 2 (SC) | | | | |
| Infected | | | | |
| G2 (POSITIVE CTRL) | 9000 | 0/10 | 8/8 | 6 |
| rMOMP L2 + 3D-MPL/DQ at week | | | | |
| 0(SC) | | | | |
| rMOMP L2 + 3D-MPL/DQ at week | | | | |
| 2(SC) | | | | |
| Sham-infected | | | | |
| G3 | 1270 | 9/10 | 7/9 | 3.3 |
| rMOMP L2 + 3D-MPL/DQ at week | | | | |
| 0(SC) | | | | |
| rMOMP L2 + 3D-MPL/DQ at week | | | | |
| 2(IN) | | | | |
| Infected | | | | |
| G4 | 1780 | 9/10 | 8/10 | 3.9 |
| rMOMP L2 + 3D-MPL/DQ at week | | | | |
| 0(SC) rMOMP L2 + mLT at week 2 | | | | |
| (IN) | | | | |
| Infected | | | | |
| G4 | 780 | 5/10 | 5/9 | 2 |
| rMOMP L2 + 3D-MPL/DQ at week | | | | |
| 0(SC) rMOMP L2 + mLT at week 2 | | | | |
| (IR) | | | | |
| Infected | | | | |

SC: subcutaneous
IN: intra-nasal
IR: intra-rectal

When compared with the positive control sham-infected group, fertility in the negative control group was nearly completely abolished, indicating the specific effect of the Chlamydial infection. Fertility of the mucosally treated groups revealed that subcutaneous priming with rMOMPL2 combined with 3D-MPL/DQ followed by a mucosal boosting afforded protection against infertility. Intranasal boosting with MOMPL2 combined with 3D-MPL/DQ or mLT afforded similar protection in terms of F and N values (groups 3 and 4, Table 10). Intra-rectal boosting also conferred protection against infertility, although to a lesser extent than the intranasal boosting.

1. A vaccine composition comprising a major outer membrane protein (MOMP) from Chlamydia in conjunction with a mucosal adjuvant, which induces a MOMP antigen specific TH1-like immune response.

2. A vaccine as claimed in claim 1 wherein the outer membrane protein is selected from the group of: serovar D to K and L.

3. A vaccine as claimed in claim 1 wherein the outer membrane is selected from the group of: F, L2, D and E.

4. A vaccine as claimed in claim 1 additionally comprising a Chlamydia MOMP protein from a different serovar, selected from the group of: serovar B, Ba, D, E, L1, F, G, K, L3, A, C, H, I and J.

5. A vaccine as claimed in claim 1 wherein the adjuvant is selected from the group comprising a combination of QS21 and 3 De-O-acylated monophosphoryl lipid A (3D-MPL), mutated heat-labile enterotoxin (mLT) or cholera toxin (CT).

6. A vaccine as claimed in claim 5 wherein QS21 additionally comprises a sterol.

7. A vaccine as claimed in claim 6 wherein the sterol is cholesterol.

8. A vaccine as claimed in claim 7 wherein QS21 is associated with a cholesterol containing liposome.

9. A vaccine as claimed in claim 5 wherein the mucosal adjuvant is LT holotoxin where arginine at position 192 is substituted with glycine (mLT R192 G).

10. A vaccine as claimed in claim 1 wherein the MOMP is the full length mature protein, devoid of the signal sequence.

11. A vaccine as claimed in claim 1 adapted for oral, or intranasal administration.

12. A vaccine as claimed in claim 1 adapted for systemic administration.

13. A delivery device pre-filled with the vaccine of claim 1, said device being designed to administer the vaccine systemically.

14. A vaccine as claimed in claim 1 wherein the outer membrane protein is produced in E. coli by recombinant DNA technology.

15. A process for the production of a vaccine comprising admixing a mucosal adjuvant with a MOMP from Chlamydia.

16. A method of inducing heterotypic prophylaxis of Chlamydia infection comprising administering to a patient a safe and effective amount of a vaccine composition of claim 1.

17. A method of inducing heterotypic prophylaxis of Chlamydia induced infertility comprising administering to a patient a safe and effective amount of a vaccine composition of claim 1.
